(19) United States
(12) Patent Application Publication
(10) Pub. No.: US 2025/0170358 A1
(43) Pub. Date: May 29, 2025

(54) GAS PATH SYSTEM AND VENTILATOR

(71) Applicant: RESVENT MEDICAL TECHNOLOGY CO., LTD, Shenzhen (CN)

(72) Inventors: Wengui ZHANG, Shenzhen (CN); Rongrong ZHU, Shenzhen (CN)

(21) Appl. No.: 18/897,471

(22) Filed: Sep. 26, 2024

(30) Foreign Application Priority Data
Nov. 27, 2023 (CN) 202323216018.9

(51) Int. Cl. A61M 16/10; A61M 16/00; A61M 16/08

(52) U.S. Cl. CPC A61M 16/105; A61M 16/022; A61M 16/0816; A61M 2205/3331; A61M 2205/42

(57) ABSTRACT

a gas path system and a ventilator are provided. The gas path system includes a high-pressure oxygen control module, a turbine module, and a main branch gas path module. The high-pressure oxygen control module transmits oxygen to the turbine module through a first gas channel extending straightly. The turbine module mixes air and the oxygen through a turbine and output mixed gas to the main branch gas path module. The main branch gas path module transmits the mixed gas to the patient through the second gas channel extending straightly. In the gas path system, through setting the first gas channel extending straightly and the second gas channel first gas channel extending straightly, a gas path structure is simplified, which avoids a phenomenon of layered cross-connection of pipelines, reduces a gas resistance in the gas path, keeps a gas flow rate uniform, and improves comfort of the patient.

### CROSS-REFERENCE TO RELATED APPLICATION

This application claims priority to Chinese Patent Application No. 202323216018.9, titled “GAS PATH SYSTEM AND VENTILATOR” and filed to the China National Intellectual Property Administration on Nov. 27, 2023, the entire contents of which are incorporated herein by reference.

### TECHNICAL FIELD

The present disclosure relates to a technical field of medical devices, and in particular to a gas path system and a ventilator.

### BACKGROUND

A ventilator is a medical device that contacts a patient's face through a mask or a nasal mask to assist the patient in breathing. The ventilator is simple to operate and has a wide range of indications. The ventilator is widely used in auxiliary treatment of respiratory diseases such as sleep apnea syndrome and severe asthma.

However, in the prior art, the ventilator has problems such as complex gas path structure, layered cross-connection of pipelines, and many right-angle pipelines. These problems may cause a resistance of the gas path of the ventilator to be large and an airflow velocity to be uneven, thus affecting comfort of the patient.

### SUMMARY

In order to at least overcome above-mentioned deficiencies in the prior art, the present disclosure aims to provide a gas path system and a ventilator.

In a first aspect, the present disclosure provides a gas path system. The gas path system comprises a high-pressure oxygen control module, a turbine module, and a main branch gas path module.

The high-pressure oxygen control module is configured to transmit oxygen. The high-pressure oxygen control module comprises a first gas channel with a gas channel extending straightly. A first end of the first gas channel is connected to the turbine module. A second end of the first gas channel is connected to an oxygen supply source.

Thee turbine module comprises a housing, a turbine disposed in the housing, a first gas inlet, a second gas inlet, and a gas outlet. The first gas inlet is connected to the first gas channel. The second gas inlet is connected to the air supply source. The turbine is configured to mix air and oxygen and output mixed gas from the gas outlet.

The main branch gas path module is configured to connect the turbine module and an air suction pipeline for a patient. The main branch gas path module comprises a second gas channel. The second gas channel extends straightly and is connected to the gas outlet.

In one optional embodiment, the high-pressure oxygen control module comprises a proportional valve, a first rectifying piece, and a first flow sensor. An outlet of the proportional valve is connected to the first gas channel. The first rectifying piece and the first flow sensor are disposed in the first gas channel. A flow direction of the oxygen in the first rectifying piece and a flow direction of the oxygen in the first flow sensor are parallel to an extending direction of the first gas channel.

The proportional valve is configured to adjust a current to control an oxygen flow, and is configured to transmit the oxygen to the first rectifying piece. The first rectifying piece is configured to rectify the oxygen to make the oxygen have a uniform flow rate. The first flow sensor is configured to measure the oxygen flow transmitted by the high-pressure oxygen control module to the turbine module.

In one optional embodiment, cross sections of the first rectifying piece perpendicular to the first gas channel increases in a direction close to the turbine module. A density of holes on the first rectifying piece increases in a direction close to the turbine module.

In one optional embodiment, the housing comprises a first chamber and a second chamber communicated with the first chamber. The first gas inlet and the second gas inlet are defined in the first chamber. The turbine and the gas outlet are disposed in the second chamber.

The turbine module further comprises damping noise reduction pieces and a noise reduction and sound absorption chamber. The noise reduction and sound absorption chamber is closed. The damping noise reduction pieces are configured to form the noise reduction and sound absorption chamber, and the damping noise reduction pieces are configured to absorb and attenuate noise.

The noise reduction and sound absorption chamber is configured to reduce the noise. The noise reduction and sound absorption chamber comprises a first noise reduction and sound absorption chamber and a second noise reduction and sound absorption chamber. The first noise reduction and sound absorption chamber and the second noise reduction and sound absorption chamber are oppositely disposed on two sides of the second gas inlet. The first noise reduction and sound absorption chamber is connected to the first gas inlet.

In one optional embodiment, the damping noise reduction pieces comprise support pieces and gridding cloths. The support pieces are disposed on two opposite inner walls of the first chamber. Each of the gridding cloths is spread on a corresponding one of the support pieces. The first noise reduction and sound absorption chamber and the second noise reduction and sound absorption chamber disposed opposite to the first noise reduction and sound absorption chamber are formed by the support pieces disposed on the two opposite inner walls of the first chamber and the gridding cloths respectively spread on the support pieces.

In one optional embodiment, the turbine module further comprises a filter. The filter is connected to the air supply source and the second gas inlet. The filter is configured to filter impurities in the air.

In one optional embodiment, the main branch gas path module comprises second rectifying pieces, a second flow sensor, an oxygen concentration sensor, and an air suction connector. The second rectifying pieces define the second gas channel. The second rectifying pieces are configured to rectify the mixed gas to make the mixed gas have a uniform flow rate. The second flow sensor is configured to measure a gas flow provided by the main branch gas path module to the air suction connector. The oxygen concentration sensor is configured to measure an oxygen concentration in the mixed gas provided by the main branch gas path module for the air suction connector. The air suction connector is configured to connect the air suction pipeline and the main branch gas path module.

In one optional embodiment, the second rectifying pieces are disposed at two ends of a gas path of the second flow sensor. The second rectifying pieces comprise etching dense porous filter screens. A density of holes on each of the etching dense porous filter screens decreases from a center portion to a periphery thereof.

In one optional embodiment, the first flow sensor and the second flow sensor comprise bidirectional flow sensors. The bidirectional flow sensors comprise gas path channels, throttling pieces, rectifying pieces, and detection pieces. The throttling pieces are respectively disposed in middle portions of the gas path channels. Each two of the detection pieces are symmetrically disposed on the two sides of each of the throttling pieces.

Each of the throttling pieces is configured to change a flow cross-sectional area of a gas flow beam so as to generate a pressure difference between the gas flow beam and a changed gas flow beam. Each of the rectifying pieces is disposed on an inner wall of a corresponding gas path channel, and is configured to suppress generation of gas turbulence. Each two of the detection pieces are configured to measure gas pressures on the two sides of each of the throttling pieces, and obtain a corresponding gas flow according to a gas pressure difference between the gas pressures on two sides of each of the throttling pieces.

In a second aspect, the present disclosure provides a ventilator, and the ventilator comprises the gas path system according to any one of embodiments mentioned above.

The present disclosure provides the gas path system and the ventilator. The gas path system comprises the high-pressure oxygen control module, the turbine module, and the main branch gas path module. The high-pressure oxygen control module is configured to transmit the oxygen to the turbine module through the first gas channel extending straightly. The turbine module is configured to mix the air and the oxygen through the turbine and output the mixed gas to the main branch gas path module. The main branch gas path module is configured to transmit the mixed gas to the patient through the second gas channel extending straightly. In the gas path system, through setting the first gas channel extending straightly and the second gas channel first gas channel extending straightly, a gas path structure is simplified, which avoids a phenomenon of layered cross-connection of pipelines and right-angle pipelines, reduces a gas resistance in the gas path, keeps a gas flow rate uniform, and improves comfort of the patient.

### BRIEF DESCRIPTION OF DRAWINGS

In order to clearly describe technical solutions in the embodiments of the present disclosure, the following will briefly introduce the drawings that need to be used in the description of the embodiments. Apparently, the drawings in the following description are merely some of the embodiments of the present disclosure, do not limit the protection scope. Those skilled in the art are able to obtain other drawings according to the drawings without contributing any inventive labor.

FIG. 1 is a structural schematic diagram of a gas path system according to one embodiment of the present disclosure.

FIG. 2 is a partial structural schematic diagram of the gas path system according to one embodiment of the present disclosure.

FIG. 3 is another partial structural schematic diagram of the gas path system according to one embodiment of the present disclosure.

FIG. 4 is another partial structural schematic diagram of the gas path system according to one embodiment of the present disclosure.

In the drawings:
10—gas path system; 110—high—pressure oxygen control module; 120—turbine module; 130—main branch gas path module; 111—proportional valve; 112—first rectifying piece; 113—first flow sensor; 121—housing; 1211—first chamber; 1212—second chamber; 122—turbine; 123—damping noise reduction piece; 1231—support piece; 1232—gridding cloth; 124—noise reduction and sound absorption chamber; 1241—first noise reduction and sound absorption chamber; 1242—second noise reduction and sound absorption chamber; 125—filter; 131—second rectifying piece; 132—second flow sensor; 133—oxygen concentration sensor; 134—air suction connector.

### DETAILED DESCRIPTION

In order to make objectives, technical solutions, and advantages of the embodiments of the present disclosure clear, technical solutions in the embodiments of the present disclosure will be described clearly and completely in conjunction with the accompanying drawings in the embodiments of the present disclosure. Obviously, the described embodiments are only a part of the embodiments of the present disclosure, rather than all of the embodiments. The components of the embodiments of the present disclosure generally described and illustrated in the figures herein may be disposed and designed in a variety of different configurations.

Therefore, the following detailed description of the embodiments of the present disclosure provided in the accompanying drawings is not intended to limit the protection scope of the present disclosure, but merely represents selected embodiments of the present disclosure. Based on the embodiments in the present disclosure, all other embodiments obtained by those skilled in the art without creative work are within the protection scope of the present disclosure.

It should be noted that similar reference numerals and letters refer to similar items in the following drawings, and therefore, once an item is defined in one figure, it does not need to be further discussed in the subsequent figures.

It should be understood in the description of the present disclosure that terms such as “upper”, “lower”, etc. indicate direction or position relationships shown based on the drawings, and are only intended to facilitate the description of the present disclosure and the simplification of the description rather than to indicate or imply that the indicated device or piece must have a specific direction or constructed and operated in a specific direction, and therefore, shall not be understood as a limitation to the present disclosure. In addition, terms such as “first” and “second” are only used for the purpose of description, rather than being understood to indicate or imply relative importance.

It should be noted in the description of the present disclosure that, unless otherwise regulated and defined, terms such as “installation”, “bonded”, and “connection” shall be understood in broad sense, and for example, may refer to fixed connection or detachable connection or integral connection; may refer to mechanical connection or electrical connection; and may refer to direct connection or indirect connection through an intermediate medium or inner communication of two pieces. For those of ordinary skill in the art, the meanings of the above terms in the present disclosure may be understood according to concrete conditions.

The embodiments, implementations and related technical features of the present disclosure can be combined and replaced with each other without conflict.

A gas path system 10 provided in embodiments of the present disclosure are applied to, but is not limited to, a ventilator to transmit mixed gas of air and oxygen to ensure a normal operation of the ventilator and safety of a patient.

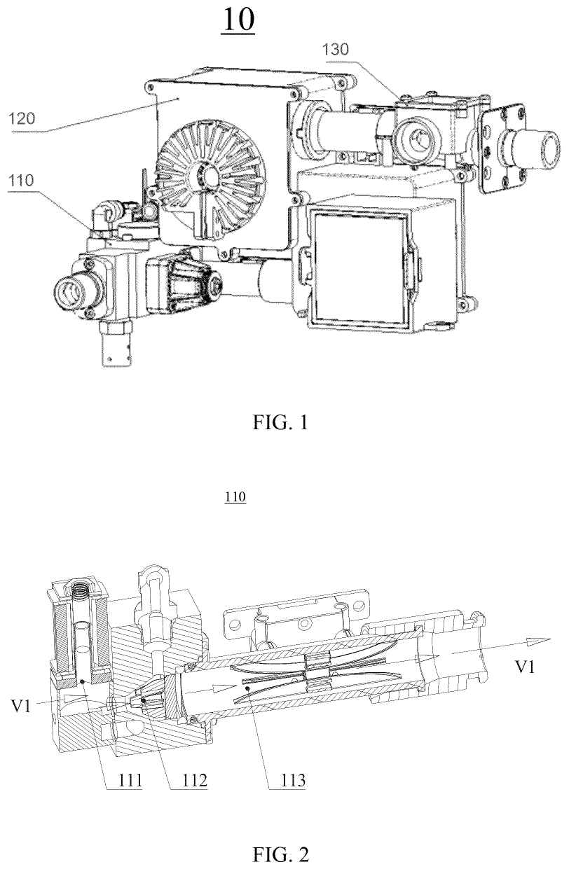

As shown in FIG. 1, the present disclosure provides a gas path system 10. The gas path system 10 comprises a high-pressure oxygen control module 110, a turbine module 120, and a main branch gas path module 130.

The high-pressure oxygen control module 110 is configured to transmit the oxygen. The high-pressure oxygen control module 110 comprises a first gas channel extending straightly to reduce a gas resistance in an oxygen supply path (i.e., the first gas channel). A first end of the first gas channel is connected to the turbine module 120. A second end of the first gas channel is connected to an oxygen supply source. Thee turbine module 120 comprises a housing 121, a turbine disposed in the housing 121, a first gas inlet 126, a second gas inlet 127, and a gas outlet 128. The first gas inlet 126 is connected to the first gas channel. The second gas inlet 127 is connected to the air supply source. The turbine 122 is configured to mix the air and the oxygen and output mixed gas from the gas outlet 128, so as to provide fresh air source for the patient. The main branch gas path module 130 is configured to connect the turbine module 120 and an air suction pipeline for the patient. The main branch gas path module 130 comprises a second gas channel. The second gas channel extends straightly and is connected to the gas outlet 128.

In the embodiment, the high-pressure oxygen control module 110 transmits the oxygen to the turbine module 120 through the first gas channel extending straightly. The turbine module 120 mixes the air and the oxygen through the turbine 122 and output the mixed gas to the main branch gas path module 130. The main branch gas path module 130 transmits the mixed gas to the patient through the second gas channel extending straightly. In the gas path system 10, through setting the first gas channel extending straightly and the second gas channel first gas channel extending straightly, a gas path structure is simplified, which avoids a phenomenon of layered cross-connection of pipelines and bending of right-angle pipelines, reduces a gas resistance in the gas path, keeps a gas flow rate uniform, and improves comfort of the patient.

In one optional embodiment, as shown in FIG. 2, the high-pressure oxygen control module 110 comprises a proportional valve 111, a first rectifying piece 112, and a first flow sensor 113. An outlet of the proportional valve 111 is connected to the first gas channel. The first rectifying piece 112 and the first flow sensor 113 are disposed in the first gas channel. A flow direction of the oxygen in the first rectifying piece 112 and a flow direction of the oxygen in the first flow sensor 113 (the direction along V1 shown in FIG. 2) are parallel to an extending direction of the first gas channel. The proportional valve 111 is configured to adjust a current magnitude, control the oxygen flow, and transmit the oxygen to the first rectifying piece 112. The first rectifying piece 112 is configured to rectify the oxygen to make the oxygen have a uniform flow rate. The first flow sensor 113 is configured to measure the oxygen flow transmitted by the high-pressure oxygen control module 110 to the turbine module 120. Specifically, the first flow sensor 113 comprises a first joint and a second joint that are disposed on two opposite ends of the first flow sensor 113. The first joint is connected to the first rectifying piece 112, and the second joint is connected to the turbine module 120.

In above structures, a size of an opening of the proportional valve 111 is controlled by adjusting a current of the proportional valve 111 to realize a purpose of adjusting the flow rate of the oxygen. After the oxygen enters the first gas channel straightly extended from the proportional valve 111, then the oxygen flows through the first rectifying piece 112 and the first flow sensor 113 and enters the turbine module 120. The first rectifying piece 112 rectifies the oxygen, increase a flow velocity of the oxygen, and makes the oxygen being uniformly distributed, so as to improve measurement accuracy of the first flow sensor 113.

Furthermore, cross sections of the first rectifying piece 112 perpendicular to the first gas channel increases in a direction close to the turbine module 120. A density of holes on the first rectifying piece 112 increases in a direction close to the turbine module 120 so as to rectify the oxygen. Therefore, the oxygen is uniformly distributed. Specifically, the oxygen has a relatively high flow velocity when entering the first rectifying piece 112 from a valve port of the proportional valve 111. Since the cross sections of the first rectifying piece 112 perpendicular to the first gas channel increases in the direction close to the turbine module 120, the flow velocity of the oxygen gradually decreases during a flow process while reducing an occurrence of turbulence, which improves uniformity and stability of the oxygen, thereby improving the measurement accuracy of the first flow sensor 113.

The inventors found in actual research processes that when a gas path structure of a conventional ventilator is complex, pipelines are layered and cross-connected, and there are many right-angle pipelines, the conventional ventilator may frequently vibrate and generate noise during gas flow. In order to reduce generation of noise and vibration and improve the comfort of the patient, the gas path system 10 provided in the embodiments of the present disclosure further comprises following noise reduction design.

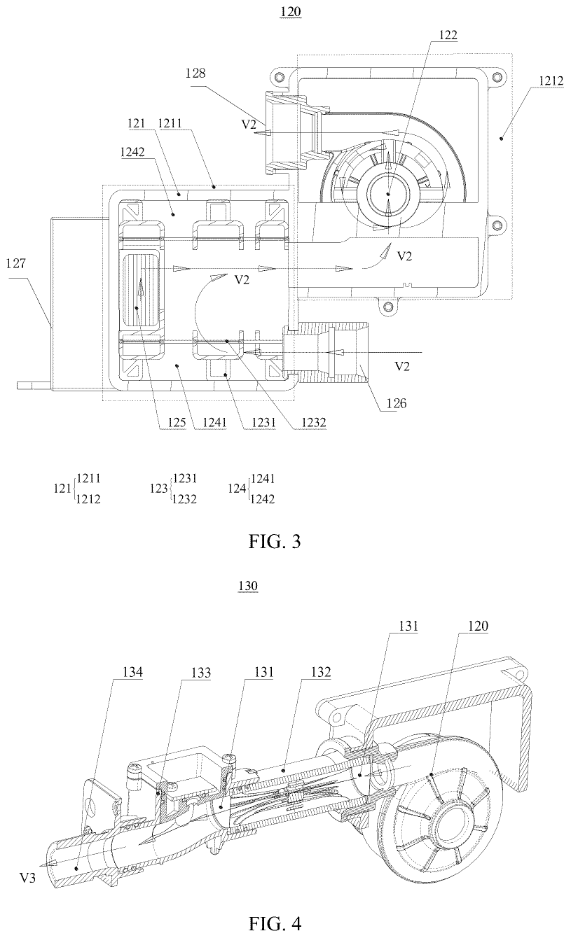

In one optional embodiment, as shown in FIG. 3, the housing 121 comprises a first chamber 1211 and a second chamber 1212 communicated with the first chamber 1211. The first gas inlet 126 and the second gas inlet 127 are defined in the first chamber 1211. The turbine 122 and the gas outlet 128 are disposed in the second chamber 1212. The turbine module 120 further comprises damping noise reduction pieces 123 and a noise reduction and sound absorption chamber 124. The noise reduction and sound absorption chamber 124 is closed. The damping noise reduction pieces 123 not only form the noise reduction and sound absorption chamber 124, but also absorb and attenuate noise. Exemplarily, the damping noise reduction pieces 123 are porous sound absorbing material. The noise reduction and sound absorption chamber 124 is configured to reduce noise. Specifically, sound waves expand and contract in the noise reduction and sound absorption chamber 124. That is, the sound waves interfere and cancel each other in the noise reduction and sound absorption chamber 124, thereby reducing the noise. In order to improve a noise-reduction effect, the noise reduction and sound absorption chamber 124 comprises a first noise reduction and sound absorption chamber 1241 and a second noise reduction and sound absorption chamber 1242. The first noise reduction and sound absorption chamber 1241 and the second noise reduction and sound absorption chamber 1242 are oppositely disposed on two sides of the second gas inlet 127. The first noise reduction and sound absorption chamber 1241 is connected to the first gas inlet 126. In addition, in order to improve the noise-reduction effect, a resonance chamber may be disposed on an outer side of the turbine module 120 to utilize a resonance effect to reduce propagation of noise of a specific frequency.

In the above structures, the oxygen enters the turbine module 120 from the first gas inlet 126, and enters the first chamber 1211 after entering the first noise reduction and sound absorption chamber 1241 for noise reduction. The air enters the turbine module 120 from the second gas inlet 127. Under an action of the turbine 122, the oxygen and the air are mixed in the first chamber 1211 and the second chamber 1212, and the mixed gas is transmitted to the main branch gas path module 130 through the gas outlet 128. A flow path is shown in FIG. 3 (see the flowing pate along V2). In addition, the turbine module 120 adopts a composite noise reduction design of resistive-expansion chambers, and cooperating with gas path designs of the first gas channel and the second channel that are straightly extended, airflow noise is effectively reduced at a gas inlet of the turbine module 120 and vibration noise generated when the turbine 122 is in operation is effectively reduced, thereby improving the comfort of the patient.

Furthermore, the damping noise reduction pieces 123 comprise support pieces 1231 and gridding cloths 1232. The support pieces 1231 are disposed on two opposite inner walls of the first chamber 1211. Each of the gridding cloths 1232 is spread on a corresponding one of the support pieces 1231. The first noise reduction and sound absorption chamber 1241 and the second noise reduction and sound absorption chamber 1242 disposed opposite to the first noise reduction and sound absorption chamber 1241 are formed by the support pieces 1231 disposed on the two opposite inner walls of the first chamber 1211 and the gridding cloths respectively spread on the support pieces 1231. Specifically, in order to facilitate processing, the support pieces 1231 are made from silicone material. In addition, the inventor found that porous foam is commonly used as a noise reduction and sound absorption material in the prior art, but the porous foam has a risk of weathering and crushing after long-term use. In order to avoid the risk, the gridding cloths 1232 with a certain strength and stability are adopted in combination with other sound absorption materials.

Furthermore, the turbine module 120 further comprises a filter 125. The filter 125 is connected to the air supply source and the second gas inlet 127. The filter 125 is configured to filter 125 impurities in the air, such as dust, particles, and other suspended matters that have a size greater than 0.3 μm.

In one optional embodiment, as shown in FIG. 4, the main branch gas path module 130 comprises second rectifying pieces 131, a second flow sensor 132, an oxygen concentration sensor 133, and an air suction connector 134. The second rectifying pieces 131 define the second gas channel (a gas flow direction thereof is along V3 shown in FIG. 4). The second rectifying pieces 131 are configured to rectify the mixed gas to make the mixed gas have a uniform flow rate. The second flow sensor 132 is configured to measure a gas flow provided by the main branch gas path module 130 to the air suction connector 134. The oxygen concentration sensor 133 is configured to measure an oxygen concentration in the mixed gas provided by the main branch gas path module 130 for the air suction connector 134. The air suction connector 134 is configured to connect the air suction pipeline and the main branch gas path module 130.

In the embodiment, the main branch gas path module 130 transmits the mixed gas from the turbine module 120 to the air suction pipeline for the patient through the second gas channel straightly extended, and the second rectifying pieces 131 rectify the mixed gas to ensure measurement accuracy of the second flow sensor 132 and the oxygen concentration sensor 133, thereby accurately monitoring gas inhaled and exhaled by the patient.

Furthermore, the second rectifying pieces 131 are disposed at two ends of a gas path of the second flow sensor 132. The second rectifying pieces 131 comprise etching dense porous filter screens. A density of holes on each of the etching dense porous filter screens decreases from a center portion to a periphery thereof, so as to rectify the mixed gas transmitted from the turbine module 120, which realizes uniform distribution of the flow rate of the mixed gas. Specifically, the mixed gas output by the turbine module 120 has a high flow velocity, and the second rectifying pieces 131 rectify the mixed gas, so that the mixed gas flows at a high speed from the center of each of the etching dense porous filter screens and gradually slows down to the periphery of each of the etching dense porous filter screens, and finally the mixed gas is enabled to have the uniform flow rate, thereby improving the detection accuracy of the second flow sensor 132 and the oxygen concentration sensor 133.

Furthermore, the first flow sensor 113 and the second flow sensor 132 comprise bidirectional flow sensors. The bidirectional flow sensors comprise gas path channels, throttling pieces, rectifying pieces, and detection pieces. The throttling pieces are respectively disposed in middle portions of the gas path channels. Each two of the detection pieces are symmetrically disposed on two sides of each of the throttling pieces. Each of the throttling pieces is configured to change a flow cross-sectional area of a gas flow beam so as to generate a pressure difference between the gas flow beam and a changed gas flow beam. Each of the rectifying pieces is disposed on an inner wall of a corresponding gas path channel, and is configured to suppress generation of gas turbulence. Each two of the detection pieces are configured to measure gas pressures on two sides of each of the throttling pieces, and obtain a corresponding gas flow according to a gas pressure difference between the gas pressures on two sides of each of the throttling pieces.

Based on the same inventive concept, the present disclosure provides a ventilator, and the ventilator comprises the gas path system 10 according to any one of embodiments mentioned above.

To sum up, the present disclosure provides the gas path system 10 and the ventilator. The gas path system 10 comprises the high-pressure oxygen control module 110, the turbine module 120, and the main branch gas path module 130. The high-pressure oxygen control module 110 is configured to transmit the oxygen to the turbine module 120 through the first gas channel extending straightly. The turbine module 120 is configured to mix the air and the oxygen through the turbine and output the mixed gas to the main branch gas path module 130. The main branch gas path module 130 is configured to transmit the mixed gas to the patient through the second gas channel extending straightly. In the gas path system 10, through setting the first gas channel extending straightly and the second gas channel first gas channel extending straightly, the gas path structure is simplified, which avoids the phenomenon of layered cross-connection of pipelines and bending of right-angle pipelines, reduces the airflow resistance in the gas path, keeps the gas flow rate uniform, and improves the comfort of the patient.

The above description is only optional embodiments of the present disclosure and is not intended to limit the present disclosure. For those skilled in the art, the present disclosure may have various modifications and variations. Any modification, equivalent replacement, improvement, etc. made within the spirit and principle of the present disclosure shall be included in the protection scope of the present disclosure.

What is claimed is:

1. A gas path system, applied to a ventilator, comprising:
a high-pressure oxygen control module,
a turbine module, and
a main branch gas path module;
wherein the high-pressure oxygen control module is configured to transmit oxygen; the high-pressure oxygen control module comprises a first gas channel extending straightly; a first end of the first gas channel is connected to the turbine module; a second end of the first gas channel is connected to an oxygen supply source;
wherein thee turbine module comprises a housing, a turbine disposed in the housing, a first gas inlet, a second gas inlet, and a gas outlet; the first gas inlet is connected to the first gas channel; the second gas inlet is connected to the air supply source; the turbine is configured to mix air and the oxygen and output mixed gas from the gas outlet;
wherein the main branch gas path module is configured to connect the turbine module and an air suction pipeline for a patient; the main branch gas path module comprises a second gas channel; the second gas channel extends straightly and is connected to the gas outlet.

2. The gas path system according to claim 1, wherein the high-pressure oxygen control module comprises a proportional valve, a first rectifying piece, and a first flow sensor; an outlet of the proportional valve is connected to the first gas channel; the first rectifying piece and the first flow sensor are disposed in the first gas channel; a flow direction of the oxygen in the first rectifying piece and a flow direction of the oxygen in the first flow sensor are parallel to an extending direction of the first gas channel;
wherein the proportional valve is configured to adjust a current to control an oxygen flow, and the proportional valve is configured to transmit the oxygen to the first rectifying piece;
wherein the first rectifying piece is configured to rectify the oxygen to make the oxygen have a uniform flow rate;
wherein the first flow sensor is configured to measure the oxygen flow transmitted by the high-pressure oxygen control module to the turbine module.

3. The gas path system according to claim 2, wherein cross sections of the first rectifying piece perpendicular to the first gas channel increases in a direction close to the turbine module; a density of holes on the first rectifying piece increases in a direction close to the turbine module.

4. The gas path system according to claim 1, wherein the housing comprises a first chamber and a second chamber communicated with the first chamber; the first gas inlet and the second gas inlet are defined in the first chamber; the turbine and the gas outlet are disposed in the second chamber;
wherein the turbine module further comprises damping noise reduction pieces and a noise reduction and sound absorption chamber; the noise reduction and sound absorption chamber is closed; the damping noise reduction pieces are configured to form the noise reduction and sound absorption chamber, and the damping noise reduction pieces are configured to absorb and attenuate noise;
wherein the noise reduction and sound absorption chamber is configured to reduce the noise; the noise reduction and sound absorption chamber comprises a first noise reduction and sound absorption chamber and a second noise reduction and sound absorption chamber; the first noise reduction and sound absorption chamber and the second noise reduction and sound absorption chamber are oppositely disposed on two sides of the second gas inlet; the first noise reduction and sound absorption chamber is connected to the first gas inlet.

5. The gas path system according to claim 4, wherein the damping noise reduction pieces comprise support pieces and gridding cloths; the support pieces are disposed on two opposite inner walls of the first chamber; each of the gridding cloths is spread on a corresponding one of the support pieces; the first noise reduction and sound absorption chamber and the second noise reduction and sound absorption chamber disposed opposite to the first noise reduction and sound absorption chamber are formed by the support pieces disposed on the two opposite inner walls of the first chamber and the gridding cloths respectively spread on the support pieces.

6. The gas path system according to claim 4, wherein the turbine module further comprises a filter; the filter is connected to the air supply source and the second gas inlet; the filter is configured to filter impurities in the air.

7. The gas path system according to claim 2, wherein the main branch gas path module comprises second rectifying pieces, a second flow sensor, an oxygen concentration sensor, and an air suction connector; the second rectifying pieces define the second gas channel;
wherein the second rectifying pieces are configured to rectify the mixed gas to make the mixed gas flow at a constant speed; the second flow sensor is configured to measure a gas flow provided by the main branch gas path module to the air suction connector;
wherein the oxygen concentration sensor is configured to measure an oxygen concentration in the mixed gas provided by the main branch gas path module for the air suction connector; the air suction connector is configured to connect the air suction pipeline and the main branch gas path module.

8. The gas path system according to claim 7, wherein the second rectifying pieces are disposed at two ends of a gas path of the second flow sensor, the second rectifying pieces comprise etching dense porous filter screens, and a density of holes on each of the etching dense porous filter screens decreases from a center portion to a periphery thereof.

9. The gas path system according to claim 7, wherein the first flow sensor and the second flow sensor comprise bidirectional flow sensors; the bidirectional flow sensors comprise gas path channels, throttling pieces, rectifying pieces, and detection pieces;
the throttling pieces are respectively disposed in middle portions of the gas path channels; each two of the detection pieces are symmetrically disposed on two sides of each of the throttling pieces;
wherein each of the throttling pieces is configured to change a flow cross-sectional area of a gas flow beam so as to generate a pressure difference between the gas flow beam and a changed gas flow beam;
wherein each of the rectifying pieces is disposed on an inner wall of a corresponding gas path channel, and is configured to suppress generation of gas turbulence;
wherein each two of the detection pieces are configured to measure gas pressures on two sides of each of the throttling pieces, and obtain a corresponding gas flow according to a gas pressure difference between the gas pressures on the two sides of each of the throttling pieces.

10. A ventilator, comprising: the gas path system according to claim 1.
